(19) United States
(12) Patent Application Publication
(10) Pub. No.: US 2025/0066760 A1
(43) Pub. Date: Feb. 27, 2025

(54) CHIMERIC ANTIGEN RECEPTOR (CAR) VECTORS AND LIBRARIES AND METHODS OF HIGH THROUGHPUT CAR SCREENING

(71) Applicant: THE CHILDREN'S HOSPITAL OF PHILADELPHIA, Philadelphia, PA (US)

(72) Inventors: John MARIS, Philadelphia, PA (US); Mark YARMARKOVICH, Philadelphia, PA (US)

(73) Assignee: THE CHILDREN'S HOSPITAL OF PHILADELPHIA, Philadelphia, PA (US)

(21) Appl. No.: 18/719,029

(22) PCT Filed: Dec. 20, 2022
(86) PCT No.: PCT/US2022/082001

§ 371 Date: Jun. 12, 2024

Related U.S. Application Data
(60) Provisional application No. 63/291,788, filed on Dec. 20, 2021.

(51) Int. Cl. C12N 15/10; C07K 14/705; C07K 14/725; C07K 16/18; C07K 16/28

(52) U.S. Cl. CPC C12N 15/1037; C07K 14/7051; C07K 14/70517; C07K 14/70521; C07K 14/70578; C07K 16/18; C07K 16/28; C07K 2317/33; C07K 2317/53; C07K 2317/622; C07K 2319/02; C07K 2319/03

(57) ABSTRACT

Expression vectors for rapid and high-throughput cloning, expression and screening of chimeric antigen receptor (CAR) constructs are provided.

### PRIORITY CLAIM

This application claims benefit of priority to U.S. Provisional Application Ser. No. 63/291,788, filed Dec. 20, 2021, the entire contents of which are hereby incorporated by reference.

This invention was made with government support under grants U54 CA232568 and R35 CA220500 awarded by National Institutes of Health. The government has certain rights in the invention.

### BACKGROUND

### I. Field

The present disclosure relates generally to the fields of molecular biology, drug discovery and immunology. It provides for engineered expression vectors useful in the screening libraries of scFv receptors for desired binding and functional properties towards difficult antigens, for use in immunotherapy development in chimeric antigen receptors (CARs), peptide centric CARs (PC-CARs) and bispecific antibodies. More specifically, these agents permit high throughput screening methods of very large numbers of candidate binders, thereby increasing the chance of identifying safe and effective constructs.

### II. Related Art

PC-CARs have the ability to target mutated or non-mutated intracellular tumor proteins presented as peptides on MHC (pMHC), opening the potential to target a plethora of novel cancer targets and expand immunotherapy to additional cancer types. Phage panning for scFv binders to pMHC is highly challenging as the peptide comprises only ˜2.5% of the pMHC complex. Cross-reactivity with MHC is unacceptable in CAR T cells, and furthermore cross-reactivity with biophysically similar peptides presented on MHC presents an additional layer of safety liability which has previously resulted in lethal cross-reactivities in clinical trials. As a result, the majority of binders identified by panning against pMHC targets are reactive against the MHC or biophysically similar non-target peptides, which would make these highly toxic as CARs or other immunotherapies.

The limited throughput of downstream screening of conventional phage panning coupled with the rarity of peptide-specific binders necessitates the screening of an excessive number of scFv clones to find antigen-specific clones. Assays used for scFvs that emerge from conventional phage screens are often decoupled from their performance in the context of CARs: 1) traditional phage approaches have thus far not yielded specific and potent scFv binders to pMHC; 2) traditional phage assays (ELISA and scFv binding studies) are usually not predictive of the function of the scFv in the context of a CAR, nor sensitive enough to detect cross-reactivity, requiring a very high number of clones to be screened in a labor-intensive process; 3) binders that appear specific in phage assays often are poorly expressed, non-functional, or cross-reactive when tested in CARs; 4) pMHC binders present a unique set of challenges as libraries are panned against a complex, of which only ˜2.5% is tumor specific. Of the 2.5%, the binding interface across the peptide needs to be maximized to increase selectivity for the antigen and minimize cross-reactivity with biophysically similar peptides on MHC.

Thus, development of a high-throughput system that pairs the scale of phage libraries with the sensitivity of CAR binding and functional assays by generating libraries of CARS derived from enriched phage libraries is highly desirable. This approach would enable high-throughput screening of binders in the context of the CAR receptor, allowing collection of highly sensitive data on on-target binding, cross-reactivity, and CAR function. Screening of all clones would allow for selection of the best CARs in each screen based on the most relevant parameters.

Additionally, a major limitation of current pMHC-targeting strategies is HLA restriction. The inventors have recently demonstrated that subsets of PC-CAR scFv clones are able to bind tumor antigens as presented by multiple different HLA allotypes. CAR libraries allow for the rapid and efficient identification of these binders to maximize patient population coverage.

### SUMMARY

The present disclosure provides a chimeric antigen receptor (CAR) expression vector encoding, in a 5′ to 3′ orientation, a promoter, a first restriction enzyme site cleaved by a first restriction enzyme, a single chain variable region antigen receptor, a second restriction enzyme site cleaved by said first restriction enzyme, a transmembrane domain and a CAR endodomain, wherein no other restriction enzyme sites for said first restriction enzyme are present in said vector. The vector may further comprise a flexible linker coding region between said second restriction enzyme site and said transmembrane domain. The vector can be part of a composition comprise a population or “library” of vectors.

Also provided is a chimeric antigen receptor (CAR) expression vector encoding, in a 5′ to 3′ orientation, a promoter, a first restriction enzyme site cleaved by a first restriction enzyme, a transmembrane domain and a CAR endodomain, wherein no other restriction enzyme sites for said first restriction enzyme are present in said vector. The vector may further comprise a flexible linker coding region between said first restriction enzyme site and said transmembrane domain, such as CD8 hinge region.

For either vector, the first restriction enzyme may be Sfi1. The promoter may be an EF1α promoter. The transmembrane domain may be derived from CD8α. The endodomain may comprise signaling domains from CD3ζ and/or 4-1BB (CD137) and/or CD28. The expression vector may further comprise an origin of replication. The expression vector may further comprise a CD8 leader sequence 5′ to said first restriction enzyme site and 3′ to said promoter.

Also provided is a method of screening a single chain fragment variable (scFv) library for binding activity comprising:
(a) providing a naïve scFv library;
(b) enriching said naïve scFv library for binding to target antigen;
(c) subcloning the scFv regions from positive binders selected in step (b) into an expression vector encoding, in a 5′ to 3′ orientation, a promoter, a first restriction enzyme site cleaved by a first restriction enzyme, a transmembrane domain and a CAR endodomain, wherein no other restriction enzyme sites for said first restriction enzyme are present in said vector;
(d) introducing the CAR library into mammalian host cells;
(e) culturing the CAR library of step (d) under conditions supporting expression of encoded CARs;
(f) incubating the host cells of step (d) with target antigen; and
(g) sorting positive host cells exhibiting CAR activation.

The target antigen may be a peptide presented on MHC. The host cell may be an immune effector cell, such as the host cells express a T cell receptor. The host cells may be NFAT Jurkat cells or primary T cells. The host cells may express GFP upon T cell receptor activation or upon CAR activation. Step (b) may comprise enriching said naïve scFv library for binders using matched and decoy pMHCs, optionally enriching a second, third or fourth time, or using a membrane protein target, optionally enriching a second, third or fourth time.

The vector may further comprise a flexible linker coding region between said first restriction enzyme site and said transmembrane domain, such as CD8 hinge region. The first restriction enzyme may be Sfi1. The promoter may be an EF1α promoter. The transmembrane domain may be derived from CD8α. The endodomain may comprise signaling domains from CD3ζ and/or 4-1BB (CD137) and/or CD28. The vector may further comprise an origin of replication. The vector may further comprise a CD8 leader sequence 5′ to said first restriction enzyme site and 3′ to said promoter.

The method may further comprising, prior to step (a), producing said naïve scFv phage library. The method may further comprise performing single-cell functional assays on the sorted positive host cells of step (f). Step (f) may comprise incubating the host cells of step (d) with cells presenting the target antigen. Step (g) may comprise sorting host cells that are positive for on-target cell killing and negative for off-target cell killing. The method may further comprise sequencing the scFv regions from host cells exhibiting activated T cell receptors. Step (b) may comprises enzyme-linked immunosorbent assays. The naïve scFv library may comprise at least 10¹⁰ unique binding sequences.

The present disclosure provides a chimeric antigen receptor (CAR) expression vector encoding, in a 5′ to 3′ orientation, a promoter, a first restriction enzyme site cleaved by a first restriction enzyme, a single chain variable region antigen receptor, a second restriction enzyme site that may or may not be cleaved by said first restriction enzyme, a transmembrane domain and a CAR endodomain, wherein no other restriction enzyme sites for said first restriction enzyme (and said second restriction enzyme site if different) are present in said vector. The expression vector may further comprise a flexible linker coding region between said second restriction enzyme site and said transmembrane domain. The expression vector may further comprise a flexible linker coding region between said first restriction enzyme site and said transmembrane domain, such as CD8 hinge region.

Also provided is a chimeric antigen receptor (CAR) expression vector encoding, in a 5′ to 3′ orientation, a promoter, a first restriction enzyme site cleaved by a first or second restriction enzyme, a transmembrane domain and a CAR endodomain, wherein no other restriction enzyme sites for said first restriction enzyme are present in said vector. The expression vector may further comprise a flexible linker coding region between said second restriction enzyme site and said transmembrane domain. The expression vector may further comprise a flexible linker coding region between said first restriction enzyme site and said transmembrane domain, such as CD8 hinge region.

For either vector, the first restriction enzyme may be Sfi1 or other unique enzyme. The promoter may be an EF1α promoter. The hinge and transmembrane domains may be derived from CD8α or CD28. The endodomain may comprise signaling domains from CD3ζ and 4-1BB (CD137), CD28, or any other T cell co-stimulatory domain. The expression vector may further comprise an origin of replication. The expression vector may further comprise a CD8 leader sequence 5′ to said first restriction enzyme site and 3′ to said promoter.

In another embodiment, there is provided a method of screening a immune receptor library for binding activity comprising:
(a) providing an immune receptor library;
(b) depleting said immune receptor library of non-specific pMHC binders;
(c) enriching said immune receptor library for binding to target antigen;
(d) subcloning the immune receptor regions from positive binders selected in step (c) into an expression vector according to claim 2-9 to produce a chimeric antigen receptor (CAR) library;
(e) introducing the CAR library into mammalian host cells;
(f) culturing the CAR library of step (e) under conditions supporting expression of encoded CARs;
(g) incubating the host cells of step (e) with target and off-target antigen and HLA-matched tissues;
(h) co-culturing the host cells of step (g) with on- and off-target cells (pMHC targets, HLA matched tissue, for membrane proteins, isogenic lines+/−target expression);
(i) sorting positive host cells exhibiting selective CAR binding and activation; and optionally;
(j) sequencing positive host cells.

The method target antigen may be a peptide presented on MHC or membrane protein, such as one that may or may not be mutated, and that may be presented by non-classic MHC (e.g., MR1). The host cell may be an immune effector cell, may express a T cell receptor, may be a Jurkat cell with or without NFAT or NF-kB-driven reporters, or a primary T cell, and/or express a fluorescent/luminescent marker upon T cell receptor activation, such as green fluorescent protein (GFP) or luciferase upon CAR activation.

Step (c) may comprise enriching said immune receptor library for binders using matched and decoy pMHCs, optionally enriching a second, third or fourth time. Step (c) may comprise enriching said immune receptor library for binders using screening against a membrane protein target, optionally enriching a second, third or fourth time. The expression vector may further comprising a flexible linker coding region between said first restriction enzyme site and said transmembrane domain, such as CD8 or CD28 hinge and transmembrane region. The first restriction enzyme may be Sfi1 or other unique enzyme. The promoter may be an EF1α promoter. The transmembrane domain may be derived from CD8α. The endodomain may comprise signaling domains from CD3ζ and 4-1BB (CD137), orCD28, ICOS, Zap70, SLP76 or other T cell signaling domains. The expression vector may further comprise an origin of replication. The expression vector further may comprise a CD8 leader sequence 5′ to said first restriction enzyme site and 3′ to said promoter.

The method may further comprise, prior to step (a), producing said immune receptor phage library or immune receptor yeast display library. The method may further comprise performing single-cell functional assays on the sorted positive host cells of step (g). Step (g) may comprise incubating the host cells of step (d) with cells presenting the target antigen. Step (h) may comprise sorting host cells that are positive for on-target cell killing and negative for off-target cell killing. The method may further comprise sequencing the immune receptor from host cells exhibiting activated T cell receptors. Step (c) may comprise enzyme-linked immunosorbent assays.

The immune library may comprise at least 10¹⁰ unique binding sequences, may comprise scFv, Vhh, Fab, monobodies, affibodies, or nanobodies, and or may be synthetic or naïve. The method may be a lossless, high-throughput screening method. Rare immune receptors may be identified. The method may comprise extension PCR with non-pComb vectors to introduce restriction sites to the immune receptor library. The immune receptor library may be derived from a primary B cell population and the target antigen is a cancer or autoimmune target. The immune receptor library is generated in single-cell droplets coupling heavy and light antibody chains through overlap PCR, optionally employing primers comprising one or more restriction sites compatible with CAR library vector ligation.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. First generation CAR development. The first-generation phage panning for pMHC binders involves co-incubating phage libraries with both tumor antigen and decoy peptide on matched MHC. Multiple rounds of panning are performed (3-4) and resulting clones are screened by ELISA to identify strong binders to tumor antigen that do not cross-react with decoy pMHC. ELISA screening is done manually in 96 well plates, significantly limiting the library diversity that can be screened, likely leaving behind many top binders.

FIG. 2. First generation CAR screens are not sensitive enough to detect cross-reactivity. Top scFv clones from ELISA screening are cloned into CAR constructs and screened for binding to tumor target and off-target pMHC. Even the most specific clones as measured by ELISA are cross-reactive when measured by more sensitive pMHC tetramer flow-based assays for CARs. Resulting CARs must be heavily optimized through a highly laborious process to reduce cross-reactivity.

FIG. 3. Combined phage/mammalian library screening strategies for pMHC binders. The inventors have developed an approach that combines the vast diversity of phage libraries with the power of in situ screening in the context of CAR libraries. The transfer of enriched phage scFvs into specially designed CAR vectors allows for multiple forms of high-throughput screening using primary T cells or Jurkat reporter systems to assess binding, function, and cross-reactivity on 10s of thousands of CARs simultaneously. Top clones can be rapidly identified, sequenced, and prioritized for preclinical development.

FIG. 4. CAR library vector design. In order to enable CAR library screening, a vector is required that is compatible with scFv phage vectors (pComb). The inventors inserted SfiI cut sites to match the phage vectors in a manner such as to optimize the addition of amino acids that could interfere with CAR binding or function. The inventors have made the CAR library vector with and without GFP. GFP vectors enable the evaluation of scFvs that lead to productive CAR clones.

FIG. 5. Agarose gels of digested CAR library vector. Fourth round VHH panning phagemid vector (left) and mammalian cell CAR library expression vector (eGFP.bbz) (right) with schematic diagram of vector design. UD refers to undigested vector, D refers to digested vector, and Lin refers to linearized vector. Equimolar volumes of phagemid vector or CAR library expression vector were incubated at 50° for five hours with either 36 U/μg or 6 U/μg SfiI, respectively (D). An undigested control was included for the phagemid vector (UD), and a linearized control was included for the CAR library expression vector (Lin). The red boxes indicate the receptor library fragments (0.375 kb, left) and empty CAR library expression vector backbone fragments (9.3 kb, right) that were excised, purified, and ligated prior to transformation.

FIGS. 6A-B. Mutation of third Sfi1 site in pJoe eGFP CAR backbone allows restriction digest of scFv region with single enzyme. (FIG. 6A) The CAR lentiviral vector contained an additional SfiI restriction site in the backbone. (FIG. 6B) Following site-directed mutagenesis, the inventors removed the cut site in the backbone and confirmed that SfiI digestion results in specific removal of the scFv. The pJoe lentiviral vector used for CAR expression was modified to remove a third SfiI site. Site-directed mutagenesis was used to mutate the third SfiI RE site to a sequence that is not recognized by the SfiI enzyme. The original and mutated pJoe eGFP CAR backbones were digested with SfiI, and the products were separated and visualized on an agarose gel. The correct product sizes (indicated to the left) demonstrate successful mutation of the third SfiI RE site.

FIG. 7. Modified CAR library vector exhibits identical binding to wild-type. CARs expressed using library vector recapitulate wild-type CAR binding. In addition to mutating the third SfiI RE site, additional amino acid sequences were added to the vector to allow cloning of the backbone with the SfiI site upstream of the scFv/V_{H}. The inventors compared binding of an scFv with known binding in the original vector and the double mutated vector (modified with the additional amino acids upstream of the scFv/V_{H} as well as removal of the third SfiI RE site). A known scFv (A7) with known binding was used in both vectors to determine what effect, if any, mutations in the backbone would have on CAR binding. Identical cross-reactivity to multiple pMHC complexes with the same HLA allotype was observed in the two vectors (original vector left, mutated vector right), suggesting incorporation of mutations necessary in the backbone to allow ligation of library scFv/V_{H} regions does not inhibit CAR binding.

FIG. 8. Peptide-centric CAR library amplification experimental overview. Phage display library screen-Phage display was used to pan for single chain variable fragments (scFv's) as well as variable heavy chains (V_{H}) using positive selection and negative selection on a PHOX2B peptide presented on A*24:02 or a CHRNA3 peptide presented on A*24:02. Multiple rounds on panning were completed to enrich for binders to either peptide-MHC complex (pMHC).

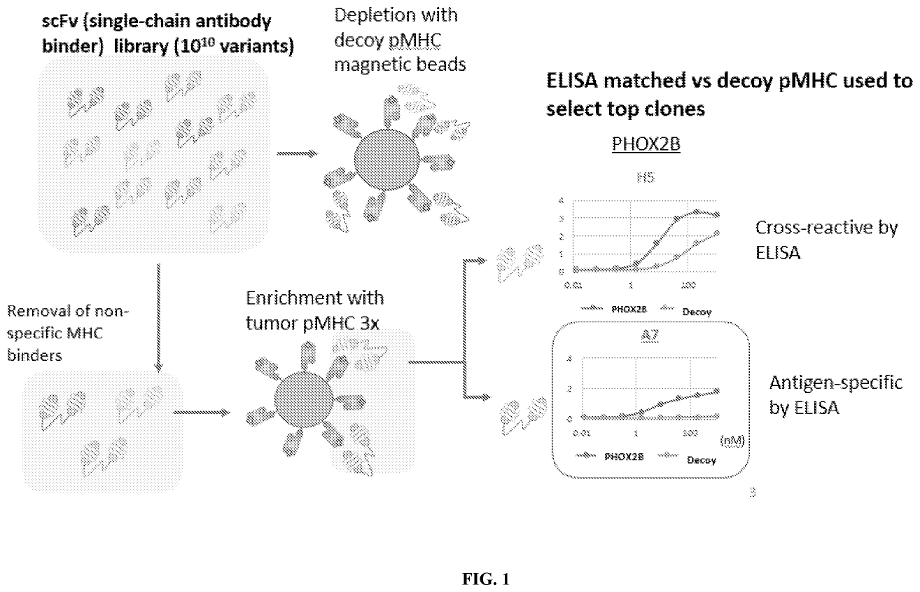

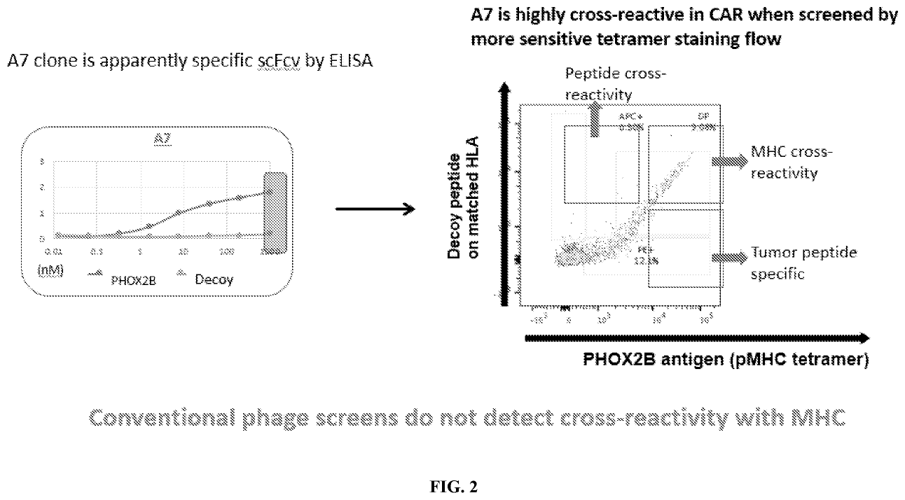

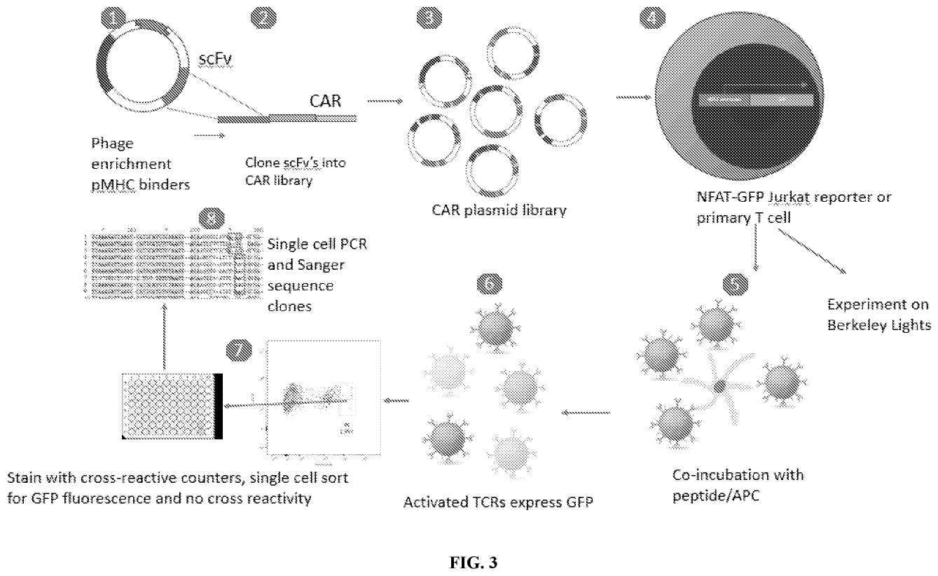

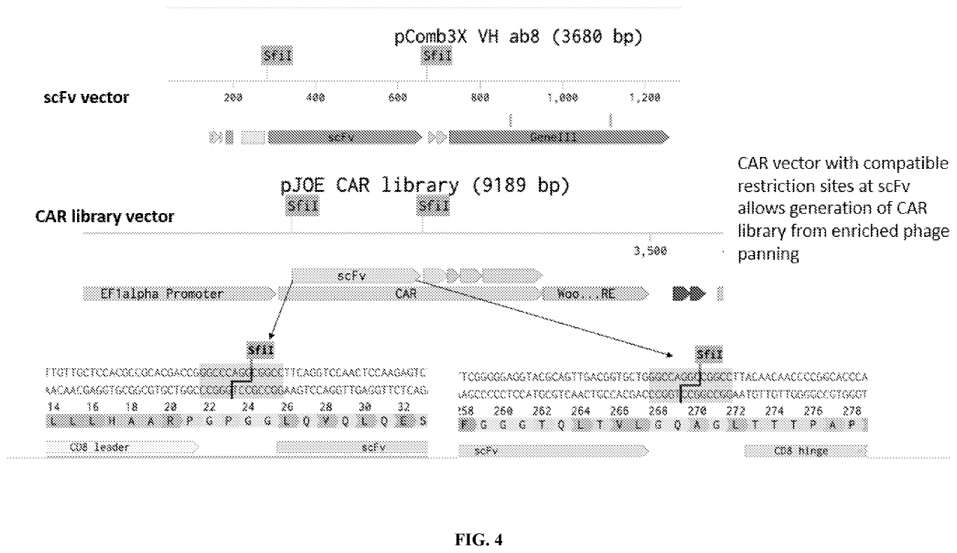

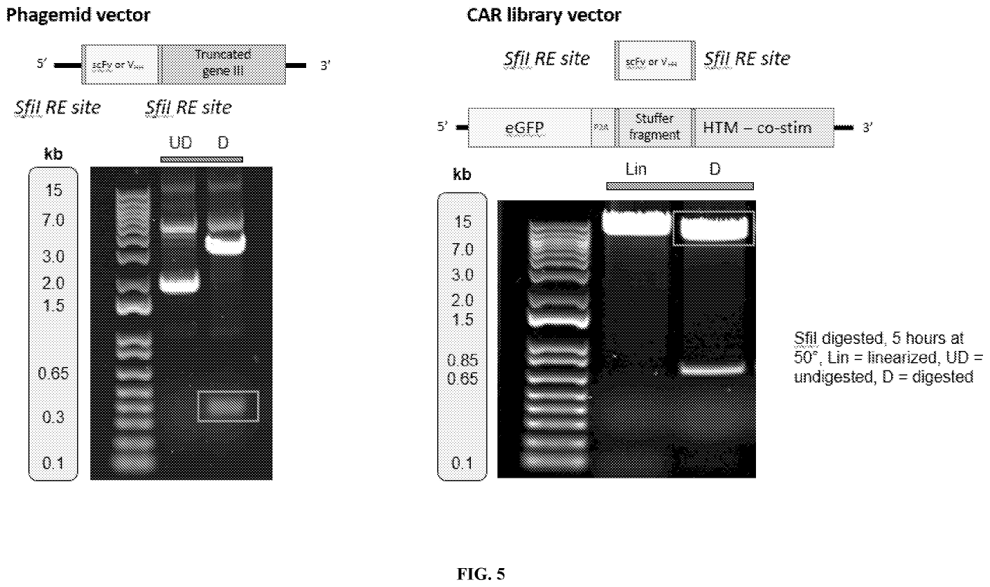

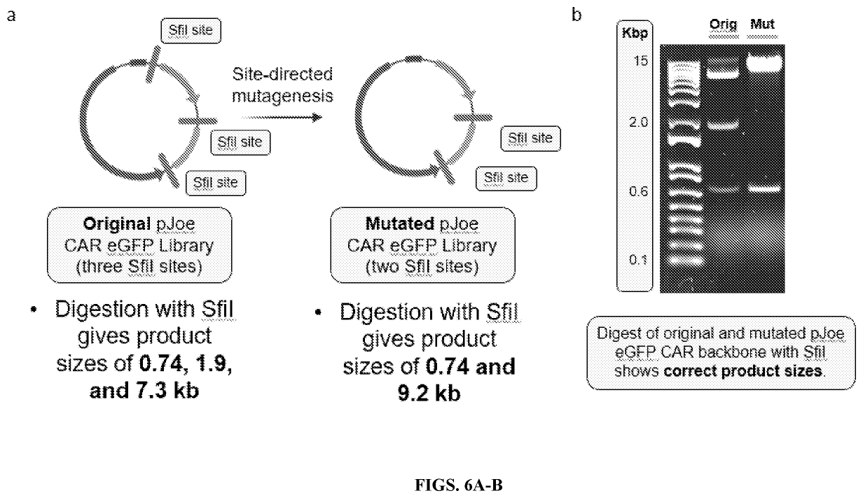

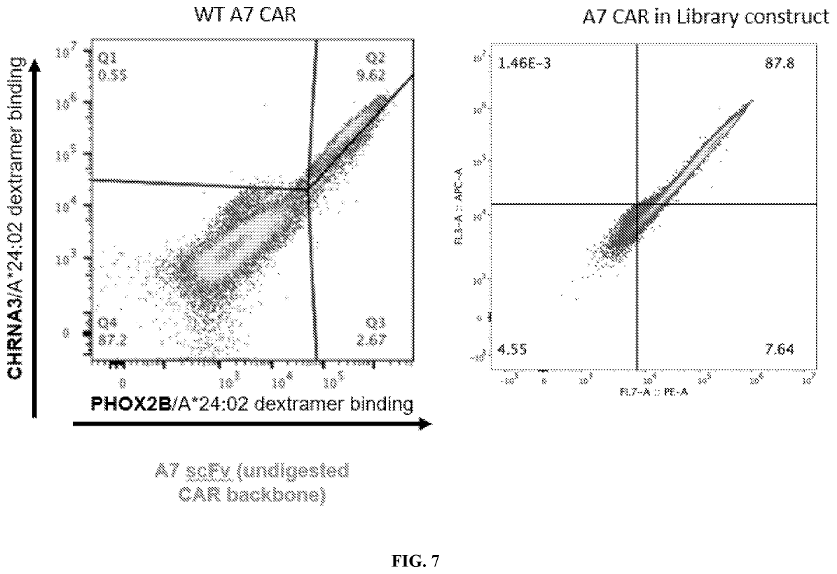

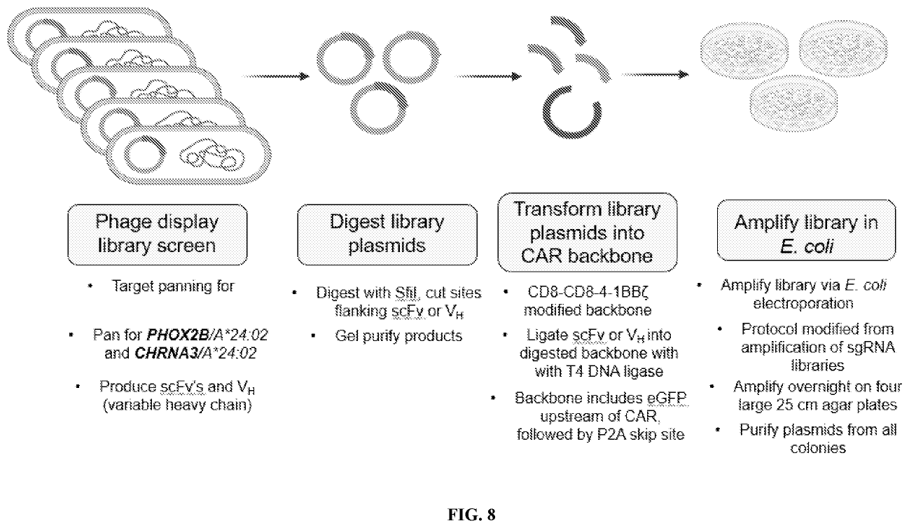

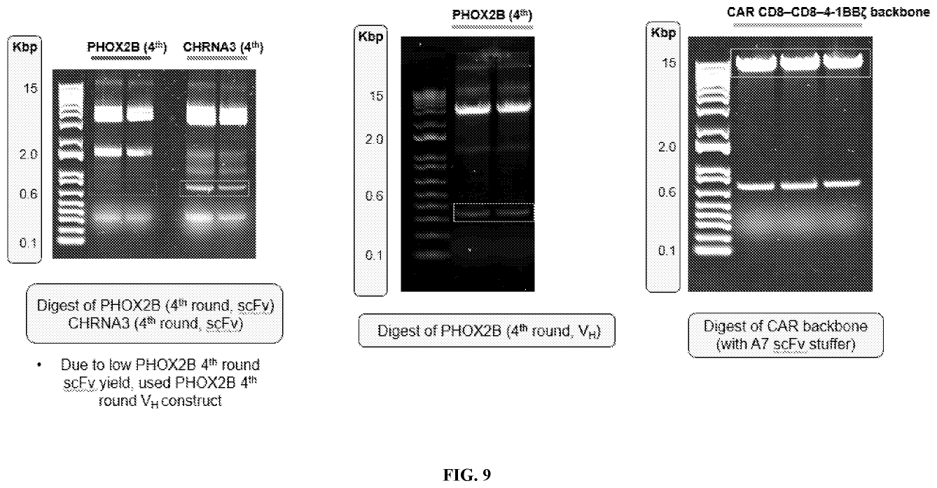

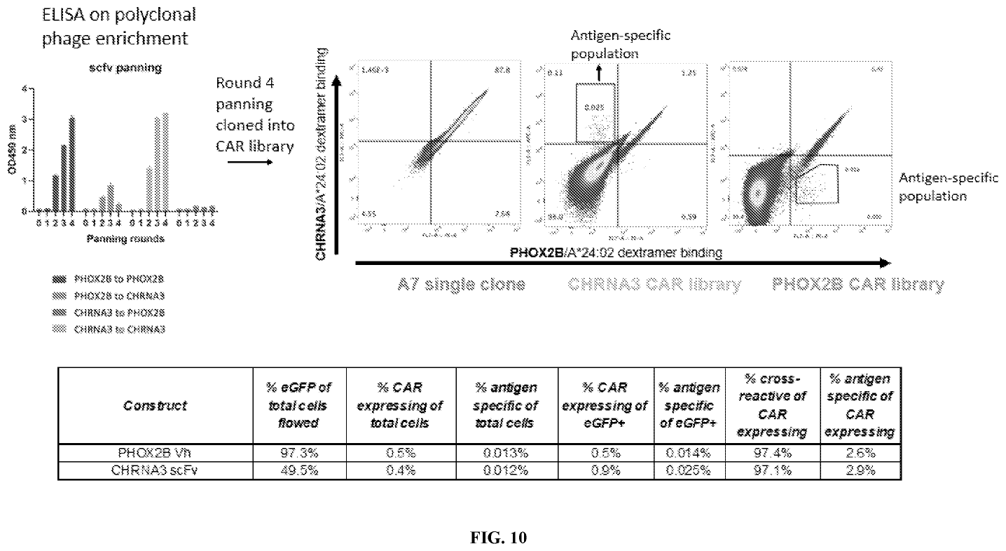

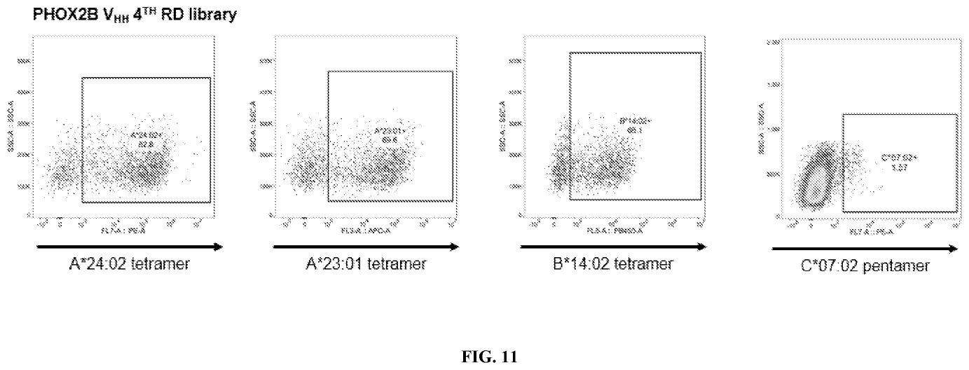

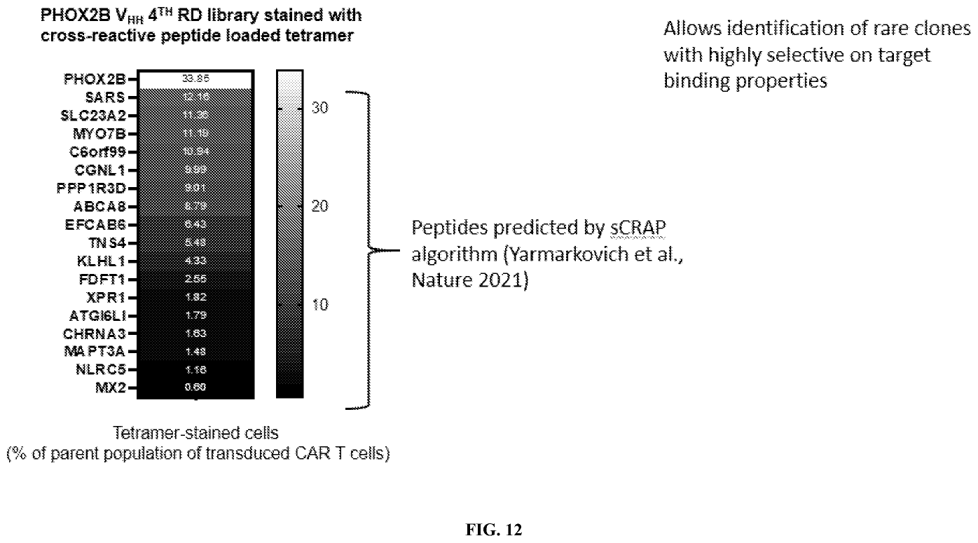

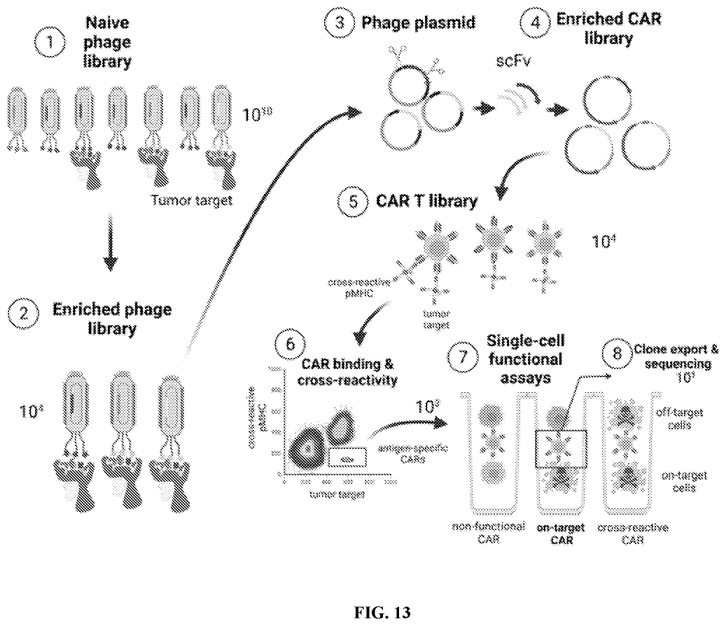

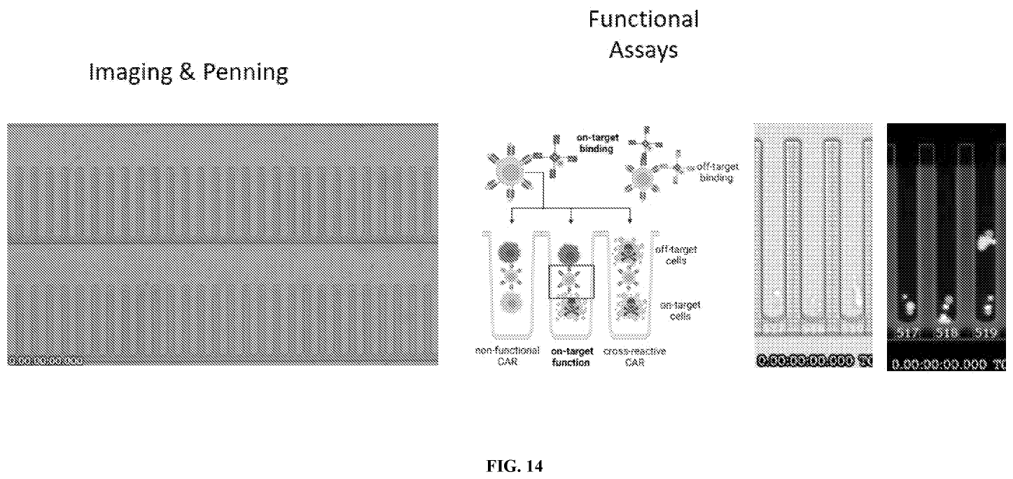

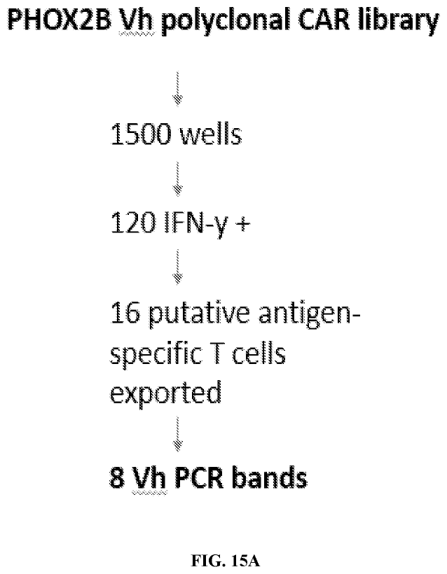

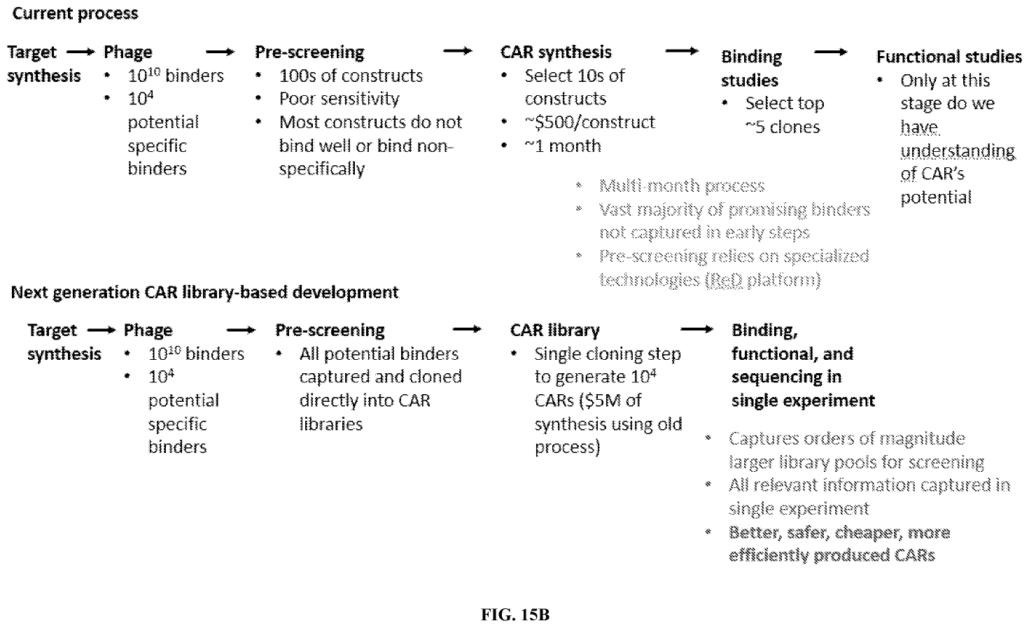

Digest library plasmids. The pCOMB3x vector plasmids containing the scFv's or V_{H} from subsequent rounds of panning were digested with the restriction enzyme (RE) SfiI, of which there are flanking cut sites on either the scFv or V_{H}. Products were separated on an agarose gel, excised, and purified. Transform library plasmids into CAR backbone. Products were ligated into a CD8 hinge/transmembrane 41BBCζ CAR modified pJOE lentiviral backbone. This backbone also co-expressed an enhanced green fluorescent protein with the CAR. Amplify library in E. coli. Ligations of library products with the CAR lentiviral backbone were transformed and amplified using E. coli. All ligation and transformation products were included to maintain library diversity within each step.

FIG. 9. Digest library plasmids with successive rounds of phage display panning decrease DNA yield of scFv/VHH obtained. The pCOMB3x vector plasmids containing the scFv's or V_{H} from subsequent rounds of panning were digested with the restriction enzyme (RE) SfiI and separated and visualized on agarose gel.

Digest of PHOX2B (4th round, scFv) CHRNA3 (4th round, scFv)-Digestion of library scFv plasmids from the fourth round of panning targeting PHOX2B peptide presented on A*24:02 or a CHRNA3 peptide presented on A*24:02 demonstrated a strong band at the expected size of the CHRNA3-targeting scFv's (0.74 Kbp) but a faint band at the expected size of the PHOX2B-targeting scFv's, suggesting the PHOX2B scFv plasmids were not fully digested. This led us to try digesting the library V_{H} plasmids from the fourth round of panning targeting PHOX2B peptide presented on A*24:02, and the products from the CHRNA3-targeting scFv digestion were used in subsequent experimental steps. Digest of PHOX2B (4^{th} round, V_{H})-Digestion of library V_{H} plasmids from the fourth round of panning targeting PHOX2B peptide presented on A*24:02 demonstrated a strong band at the expected size of the PHOX2B-targetong V_{H} (0.34 kbp). These products were used in subsequent experimental steps. Digest of CAR backbone (with A7 scFv stuffer)-Digestion of mutated CAR backbone with SfiI to remove stuffer scFv fragment demonstrated expected sizes of digested products (empty mutated CAR backbone at 9.3 kbp and stuffer scFv fragment at 0.74 kbp). The empty mutated CAR backbone was excised and gel purified before use in subsequent ligation steps with the gel purified library scFv's (targeting CHRNA3) or library V_{H}'s (targeting PHOX2B).

FIG. 10. CAR libraries enable identification of rare antigen-specific polyclonal populations for PHOX2B and CHRNA3. Following cloning of the enriched phage libraries panned against both PHOX2B and CHRNA3 pMHC, libraries were cloned into CAR library GFP vectors vectors. Screening for on- and off-target binding reveals that the vast majority of scFvs lead to non-productive binders (˜99.5%). Of these, the vast majority are cross-reactive with MHC (˜97.3%). Only ˜0.0195% of scFvs are found to be antigen-specific, suggesting that ˜5000 clones would need to be screened to identify a single peptide-centric binder, an infeasible process for conventional phage screening. This method allows the sorting and high-throughput in situ screening of this entire polyclonal antigen-specific population to rapidly identify the binders with optimal killing and specificity profiles.

FIG. 11. CAR Library allows for rapid identification of cross-HLA binders. The inventors have previously shown the ability of CARs to recognize peptides across HLA types. This required large scale screening of individual CAR constructs to find rare clones with cross-HLA binding properties. They are now able to rapidly identify the population of clones with cross HLA binding properties. The inventors demonstrate the ability to robustly identify cross-HLA binding as previously reported between HLA-A*24:02 and HLA-A*23:01 and in addition were able to identify a rare population of clones with cross-HLA binding to HLA-C*07:02, not previously found in other low throughput screens.

FIG. 12. CAR library allows for rapid and multiplexed screening of cross-reactivity. The inventors can now assess population-scale cross-reactivity against an entire panel of potentially cross-reactive peptides. They previously showed that clones that are cross-reactive against at least one of the predicted peptides are also cross-reactive against HLA matched tissue. The CAR library allows the parallel screening of 10s of thousands of binders to identify the rare clones with highly selective binding.

FIG. 13. Berkeley Lights experiment. Enriched CAR libraries transduced into T cells (5) can be stained with tetramers for tumor antigens and cross-reactive peptides on the same HLA for flow cytometry and cell sorting (6). Rare antigen-specific populations can be isolated through cell sorting or bead-based enrichment and loaded onto the Berkeley Lights Lightning instrument which allows individual T cell clones to be assayed for on- and off-target binding, on- and off-target cell killing, and cytokine release. The Lightning chip allows screening of ˜1500 antigen-specific clones in parallel, collecting data on selectivity, functionality, and persistence of all clones and allowing the rapid and efficient selection of optimal clones.

FIG. 14. CAR library screens on Lightning. CAR libraries can be screened for functional activity using optofluidic technologies such as the Berkeley Lights Lightning system. Cells can be stained for on- and off-target cross-reactivity using pMHC tetramers for tumor targets and sCRAP-predicted peptides. CAR T clones with desired binding properties can be penned in nanowells for single cell functional assays. Single CAR clones can be screened for on- and off-target killing using target tumor cells and off-target HLA-matched tissues. Rare cell clones with both desired binding and functional properties can be exported and their receptors sequenced for preclinical development.

FIGS. 15A-B. (FIG. 15A) Summary of Berkeley Lights experiment. Starting with 1500 enriched CAR clones, 8 clones are identified with antigen-specific binding and killing properties. (FIG. 15B) Comparison of current PC-CAR development process to CAR library-based development.

### DETAILED DESCRIPTION

The curative potential of chimeric antigen receptor (CAR) T cell-based cancer immunotherapies has been widely established. However, identifying new binding agents for use in CAR constructs is a time-consuming and laborious property that is hindered by the large number of potential binders that can be generated by a human immune system and the small number of high-quality binding agents with the properties necessary for development into bona fide therapeutic reagents.

The inventors have generated a CAR construct that is compatible with phage display libraries, allowing enriched phage libraries to be cloned directly into CAR constructs. These CAR libraries can then be rapidly screened for clones with desired binding and functional properties, allowing the screening of thousands of different clones in one step, rather than having to synthesize individual CAR constructs as the inventors have been doing. Currently, clones isolated from phage display are enriched, ELISAs are performed in the context of phage (giving an imperfect representation of binding in the context of a CAR), top clones are selected using irrelevant assays for sequencing and synthesized into CAR constructs. This is a long and laborious process and only allows for the selection for ˜10s of clones. The inventors' approach allows for enriched phage libraries to be cloned directly into CAR constructs, maintaining the entire diversity of the library and bypassing the need for sequencing and synthesis. The large diversity maximizes the likelihood of identifying specific and potent binders, and the direct cloning saves a vast amount of time and money. T cells transduced with these CAR libraries can rapidly be screened for on- and off-target binding as well as specific and non-specific killing.

These and other aspects of the disclosure are set out in detail below.

### I. Definitions

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, antibodies and related molecules, cell and tissue culture, molecular biology, and protein and oligo- or polynucleotide chemistry and hybridization described herein are those well-known and commonly used in the art.

Standard techniques are used for recombinant DNA, oligonucleotide synthesis, and cell culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See, e.g., B. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); and F. M. Ausubel et al., (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory (1988); and J. E. Coligan et al., (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md. (1987 and 1991); Bork et al., J Mol. Biol. 242, 309-320 (1994); Chothia and Lesk J. Mol Biol. 196:901-917 (1987), Chothia et al., Nature 342, 877-883 (1989), and/or or Al-Lazikani et al., J Mol Biol 273, 927-948 (1997).

As used herein, “antigen-binding site” shall be taken to mean a structure formed by a protein that is capable of binding or specifically binding to an antigen, such as an antibody. The antigen-binding site need not be a series of contiguous amino acids, or even amino acids in a single polypeptide chain. For example, in a Fv comprising two different polypeptide chains from an antibody, the antigen-binding site is made up of a series of amino acids of a V_{L} and a V_{H} that interact with the antigen and that are generally, however not always in one or more of the CDRs in each variable region. In some embodiments, the antigen-binding site is an antigen-binding site of an antibody. In such embodiments, the antigen-binding site may comprise one or more complementarity-determining regions or “CDRs”. In some embodiments, the antigen-binding site of an antibody comprises at least part of a V_{H} or a V_{L} or a Fv.

As used herein, the terms “complementarity-determining region” or “CDR” are used interchangeably to refer to the antigen binding regions found within the variable region of the heavy and light chain polypeptides. Generally, antibodies comprise three CDRs in each of the V_{H} (CDR H1 or H1; CDR H2 or H2; and CDR H3 or H3) and three in each of the V_{L} (CDR L1 or L1; CDR L2 or L2; and CDR L3 or L3).

As used herein, the “variable regions” and “CDRs” may refer to variable regions and CDRs defined by any approach known in the art, including combinations of approaches. According to a specific embodiment, the CDRs are determined according to Kabat et al., (supra).

As used herein “binding” or “binds” or “specifically binds” refers to an antibody: antigen mode of binding, which preferably, in the case of clinically relevant binding agents, means a K_{D} below 1 μM or below 500 nM. The binding agents of the disclosure can bind PHOX2B: pMHC complexes with a high affinity. For example, in some embodiments the binding agent can bind PHOX2B: pMHC with a dissociation constant (K_{D}) equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴. Specificity of binding is determined with reference to non-target proteins, such as for example bovine serum albumin (BSA). In some embodiments the binding agent binds PHOX2B: pMHC complexes with a dissociation constant (K_{D}) at least 2, 3, 4, 5, 10, 20, 50, 100, 200, 500, 1000, 2000, 5000, 10⁴, 10⁵ or 10⁶-fold lower than the binding agent's dissociation constant for BSA, when measured at physiological conditions. In some cases, specificity is determined by measuring binding of a binding agent to an MHC that is loaded with a non-target peptide or that is empty. In some cases, specificity is determined by measuring binding of a binding agent to the target peptide alone or the target peptide loaded on an MHC of a different allotype. In particular embodiments of the present disclosure, the binding agent is MHC-restricted which means that the binding agent binds specifically to a target peptide (e.g., PHOX2B peptide) loaded onto an MHC representative of a chosen allelic variant (e.g., HLA-A*24:02) with a dissociation constant (K_{D}) at least 2, 3, 4, 5, 10, 20, 50, 100, 200, 500, 1000, 2000, 5000, 10⁴, 10⁵ or 10⁶-fold lower than the binding agent's dissociation constant for an MHC from another allelic variant.

As used herein the phrase “chimeric antigen receptor (CAR)” refers to a recombinant or synthetic molecule which combines antibody-based specificity for a desired antigen with a T cell receptor-activating intracellular domain to generate a chimeric protein that exhibits cellular immune activity to the specific antigen.

As used herein the phrase “T Cell Receptor” or “TCR” refers to soluble and non-soluble forms of recombinant T-cell receptor

As used herein, a “T-cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell.

As used herein a “T Cell Receptor-like antibody (TCRL” or “peptide-centric CAR (PC-CAR)” refers to an antibody which binds an MHC displaying an HLA-restricted peptide antigen. Binding of the TCRL to its target typically has an MHC-restricted specificity: the TCRL does not bind the MHC in the absence of the complexed peptide, and the TCRL does not bind the peptide in an absence of the MHC. TCRLs are characterized by affinity sufficient to permit specific binding to a tumor antigen even when the TCRL is provided in a soluble, rather than membrane-bound, form. TCRLs are being developed as a new therapeutic class for targeting tumor cells and mediating their specific killing. In addition, TCRLs are valuable research reagents enabling the study of human class I peptide-MHC ligand presentation and TCR-peptide-MHC interactions. In an embodiment, the binding agent of the present disclosure is a TCRL.

As used herein the phrase “MHC (or HLA)-restricted peptide” refers to a peptide which is potentially presented on an MHC molecule. Such peptides may be identified by laboratory procedures such as Mass-Spectrometry, reverse-immunology or by in-silico analysis. An MHC (or HLA)-presented peptide refers to a peptide which is confirmed in vitro or in vivo as being presented by an MHC molecule.

The term “cancer” as used herein is defined as disease characterized by the rapid and uncontrolled growth of aberrant cells. Cancer cells can spread locally or through the bloodstream and lymphatic system to other parts of the body.

A “compound” refers to any molecule including small molecules, polypeptides, and other macromolecules. In some embodiments, a compound is a small molecular weight compound with a molecular weight of less than about 2000 Daltons.

The term “naturally-occurring” (or “native”) as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally occurring.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical linker or a disulphide bond, for example. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics.

The term “agent” is used herein to denote a chemical compound, a mixture of chemical compounds, a biological macromolecule, or an extract made from biological materials.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

### III. Binding Agents

The term “binding agent”, as used herein, refers to any molecule which is capable of binding to a target of interest, e.g., a cancer antigen. In some embodiments, the binding agent is or comprises a polypeptide. In some embodiments, the binding agents of the present disclosure recognized an antigen in the context of MHC/HLA presentation. In some embodiments, the binding agents of the present disclosure further comprise dimeric binding agents derived by splitting the single chain variable fragment (scFv) sequences into light chain and heavy chain, respectively, at the poly-G/S linker, as well as homologs or variants of such dimeric binding agents. In particular embodiments, the binding agent comprises a heavy chain and a light chain which comprises three heavy chain CDR and three light chain CDR sequences, respectively, of the present disclosure, maintaining or improving binding. In embodiments of the disclosure, the binding agent is an antibody, or antigen-binding fragment thereof, an artificial protein that is soluble (e.g., a bispecific antibody), or an artificial protein that is membrane-tethered (e.g., a chimeric antibody receptor or a TCR fusion protein).

For binding agents derived from immunoglobulin (Ig) variable domains, with the target contact surface created through the loops connecting β-strands (the complementarity-determining regions, or CDRs), the binding activity to the target may be transferable through the grafting of the CDR loops to related Ig domains (e.g., other human Ig family members) or even non-Ig B-sheet scaffolds. This is especially the case where the structure of the binding agent in complex with the target indicates that the binding is mainly contributed through one, or a few, of the 6 CDRs of the combined V_{L} and V_{H} domains. CDR grafting has been used extensively to ‘humanize’ antibodies where the CDR loops from antibodies derived from a non-human host are grafted onto a human Ig scaffold to reduce immunogenicity. Many antibodies approved for therapeutic use have been humanized through CDR transplantation from murine antibodies onto human scaffolds. Examples where the grafting of CDR loops from antibody scaffolds onto non-Ig alternative scaffolds have also been reported (Nicaise M., et al., Protein Sci 13:1882-1891 (2004); Petrovskaya L E, et al., Biochemistry (Mosc) 77:62-70 (2012); Pacheco et al., Protein Eng Des Sel 27:431-438 (2014)).

Various means of determining the K_{D} of a binding agent for its target are known, including enzyme-linked immunoabsorbance (ELISA) assays and Surface Plasmon Resonance (SPR) assays. In some cases binding affinity and specificity is determined by optical interferometry, such as with the Pall ForteBio BLItz® system, as described in Sultana A. Lee J. Curr Protoc Protein Sci, 79:19.25.1-19.25.26 (2015). Affinity of a binding agent may be determined using a soluble form of the binding agent or a membrane-tethered form, such as a chimeric antigen receptor (CAR) or T-cell receptor (TCR) fusion protein (TFP). Conversely, the pMHC complex may be tested in a soluble form or in its native, cell-membrane-bound state.

### A. Antibodies

In some embodiments, the binding agent is an antibody or antibody fragment. Suitable antibody fragments for practicing some embodiments of the disclosure include between one and three complementarity-determining region (CDRs) of an immunoglobulin light chain (referred to herein as “light chain”) and between one and three CDRs of an immunoglobulin heavy chain (referred to herein as “heavy chain”). Optionally, the binding agent comprises a variable region of a light chain, a variable region of a heavy chain, a light chain, or a heavy chain.

The identity of the amino acid residues in a particular antibody that make up a variable region or a CDR can be determined using methods well known in the art and include methods such as sequence variability as defined by Kabat et al., (See, e.g., Kabat et al., 1992 Sequences of Proteins of Immunological Interest, 5th ed., Public Health Service, NIH, Washington D.C), location of the structural loop regions as defined by Chothia et al., (see, e.g., Chothia et al., Nature 342:877-883 (1989), a compromise between Kabat and Chothia using Oxford Molecular's AbM antibody modeling software (now Accelrys®, see, Martin et al., Proc Natl Acad Sci USA. 86:9268 (1989); and world-wide-web at bioinf-org.uk/abs), available complex crystal structures as defined by the contact definition (see MacCallum et al., J. Mol. Biol. 262:732-745 (1996)), the “conformational definition” (see, e.g., Makabe et al., Journal of Biological Chemistry, 283:1156-1166 (2008)), and the IMGT method (Lefranc M P, et al., IMGT unique numbering for immunoglobulin and T cell receptor variable domains and Ig superfamily V-like domains Dev Comp Immunol 27:55-77 (2003)).

In embodiments, the binding agent is a functional antibody fragment comprising whole or essentially whole variable regions of both light and heavy chain, including but not limited to those defined as follows: (i) Fv, defined as a fragment consisting of the variable region of the light chain (VL) and the variable region of the heavy chain (VH) expressed as two chains; (ii) single chain variable fragment or single chain Fv (“scFv”), a genetically engineered single chain molecule including the variable region of the light chain and the variable region of the heavy chain, linked by a suitable polypeptide linker as a genetically fused single chain molecule; (iii) disulfide-stabilized Fv (“dsFv”), a genetically engineered antibody including the variable region of the light chain and the variable region of the heavy chain, linked by a genetically engineered disulfide bond; (iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CHI domains thereof; (v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule); (vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and (vii) single domain antibodies or nanobodies are composed of a single V_{H} or V_{L} domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988).

Antibody fragments according to some embodiments of the disclosure can be prepared by proteolytic hydrolysis of the antibody or by expression in E. coli or mammalian cells (e.g., Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein. See also Porter, R. R., Biochem J. 73:119-126 (1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In an embodiment in which the binding agent is an antibody, the heavy and light chains of an antibody of the disclosure may be full-length (e.g., an antibody can include at least one, and preferably two, complete heavy chains, and at least one, or two, complete light chains). In some embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1) or IgG4 (e.g., human IgG4). The choice of antibody type will depend on the immune effector function that the antibody is designed to elicit. In an embodiment, the binding agent elicits antibody dependent cellular cytotoxicity. In an embodiment, the binding agent elicits complement dependent cytotoxicity.

Bispecific configurations of antibodies are also contemplated herein. A bispecific monoclonal antibody (BsMAb, BsAb) is an artificial protein, or complex of proteins, that is composed of fragments of two different monoclonal antibodies and consequently binds to two different types of antigen. According to a specific embodiment the BsMAb is engineered to simultaneously bind to an effector cell (e.g., using a receptor like CD3) and a target like a tumor cell to be destroyed. Anti-CD3ζ antibodies known to the art and used for directing bispecific antibody engagement with CD3-positive effector cells include SP-34 (Pessano et al., EMBO J (1985) 4:337-344), OKT3 (Kung et al., Science (1979) 206:347-349), UCHT1 (Beverley PCL, Callard R E Eur J Immunol (1981) 11:329), 12F6 (Wong J T and Colvin R B, J Immunol (1987) 139:1369-1374), and humanized and/or affinity engineered variants of all (e.g., Shalaby et al., J Exp Med (1992) 175:217-225). Other affinity scaffolds, such as VHH domains, may also be used to engineer CD3ζ binding (e.g., WO/2015/095412). Other configurations, such as tri-specific or tetra-specific antibodies, for example, are also contemplated.

B. Single-Chain Variable Fragment (scFv)

Fv fragments comprise an association of V_{H} and V_{L} chains. This association may be noncovalent, as described in Inbar et al., Proc Natl Acad. Sci. USA 69:2659-62 (1972). Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are described, for example, by Whitlow and Filpula, Methods 2:97-105 (1991); Bird et al., Science 242:423-426 (1988); Pack et al., Bio/Technology 11:1271-77 (1993); and U.S. Pat. No. 4,946,778. The resulting polypeptides can fold back on themselves to form antigen-binding monomers, or they can form multimers (e.g., dimers, trimers, or tetramers), depending on the length of a flexible linker between the two variable domains (Kortt et al., Prot Eng 10:423 (1997); Kortt et al., Biomol Eng 18:95-108 (2001)). By combining different V_{L} and V_{H}-comprising polypeptides, one can form multimeric scFvs that bind to different epitopes (Kriangkum et al., Biomol. Eng. 18:31-40 (2001)). Techniques developed for the production of single chain antibodies include those described in U.S. Pat. No. 4,946,778; Bird, Science 242:423 (1988); Huston et al., Proc Natl Acad Sci USA 85:5879 (1988); Ward et al., Nature 334:544 (1989), de Graaf et al., Methods Mol Biol. 178:379-87 (2002). Single chain antibodies derived from binding provided herein include, but are not limited to, scFvs comprising one or more variable domain sequences, or one or more CDR sequences from one or more variable domain sequences, disclosed herein.

### C. Chimeric Antigen Receptor (CAR) and TCR Fusion Proteins (TFP)

Chimeric antigen receptors (CARs) are fusion proteins comprising antigen recognition moieties and T cell-activation domains. Exemplary CARs are provided by U.S. Pat. Nos. 8,399,645 and 7,638,325. Other exemplary recombinant receptors, including CARs, recombinant T-cell receptors (TCRs), TCR fusion proteins (TFPs), as well as methods for engineering and introducing the receptors into cells, include those described in Int'l Pat. Appl. Nos. WO2017/096329, WO2000/14257, WO2013/126726, WO2012/129514, WO2014031687, WO2013/166321, and WO2013/071154, WO2013/123061, and WO/2014055668; U.S. Pat. App. Nos. US2002131960, US2013287748, and US20130149337; U.S. Pat. Nos. 6,451,995, 7,446,190, 7,638,325, 8,252,592, 8,339,645, 8,398,282, 7,446,179, 6,410,319, 7,070,995, 7,265,209, 7,354,762, 7,446,191, 8,324,353, and 8,479,118; European Pat. App. No. EP2537416; and Sadelain et al., Cancer Discov. April 3 (4): 388-398 (2013); Davila et al., PLOS ONE 8 (4): e61338 (2013); Turtle et al., Curr. Opin. Immunol. October 24 (5): 633-39 (2012); and Wu et al., Cancer, March 18 (2): 160-75 (2012). In an embodiment, the binding agent is a TFP as described in U.S. patent Ser. No. 15/419,398.

### IV. Expression Constructs

According to the disclosure there are provided an engineered vectors that facilitate cloning and screening of scFV directly from phage libraries. Such expression vectors contain elements coding for the non-antigen binding portions of CARs along with cis-acting regulatory elements. The expression vector of some embodiments of the disclosure includes additional sequences which render this vector suitable for replication and integration in prokaryotes, eukaryotes, or preferably both (e.g., as a shuttle vector). In addition, typical cloning vectors may also contain a transcription and translation initiation sequence, transcription and translation terminator and a polyadenylation signal.

The nucleic acid construct of some embodiments of the disclosure includes a signal sequence for secretion or presentation of the binding agent from a host cell in which it is placed. Preferably the signal sequence for this purpose is a mammalian signal sequence.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. Preferably, the promoter utilized by the expression vector is active in the specific cell population transformed. Examples of cell type-specific and/or tissue-specific promoters include promoters such as albumin that is liver specific (Pinkert et al., Genes Dev. 1:268-277 (1987)), lymphoid specific promoters (Calame et al., Adv. Immunol. 43:235-275 (1988)); in particular promoters of T-cell receptors (Winoto et al., EMBO J. 8:729-733 (1989)) and immunoglobulins; (Banerji et al., Cell 33:729-740 (1983)), neuron-specific promoters such as the neurofilament promoter (Byrne et al., Proc. Natl. Acad. Sci. USA 86:5473-5477 (1989)), pancreas-specific promoters (Edlunch et al., Science 230:912-916 (1985)) or mammary gland-specific promoters such as the milk whey promoter (U.S. Pat. No. 4,873,316 and European Application Publication No. EP0264166). In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Enhancer elements can stimulate transcription up to 1,000-fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the disclosure include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983.

Polyadenylation sequences can also be added to the expression vector in order to increase the efficiency of TCRL mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the disclosure include those derived from SV40.

In addition to the elements already described the expression vector of some embodiments of the disclosure may contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein. Such cells are typically selected for high expression of recombinant proteins (e.g., bacterial, plant or eukaryotic cells e.g., CHO, HEK-293 cells), but may also be host cells having a specific immune effector activity (e.g., T cells or NK cells) when for instance the CDRs of the TCRL are implanted in a T Cell Receptor or CAR transduced in said cells which are used in adoptive cell therapy.

Next, the CAR library is introduced into mammalian host cells that are then cultured under conditions supporting expression of encoded CARs. The host cells expressing the CAR are then contacted with target antigen positive host cells exhibiting CAR activation are then identified using a variety of different approaches. Once these cells are identified, the binding region can be sequenced and further developed.

### V. Screening Methods

Screening methodologies are well known in the art. The initial step will be generation of a first library, often called a naïve library, of antigen binding sequences. Phage display libraries can be employed for rapid screening. Such libraries may be enhanced for strong/selective binders using various methods.

As discuss above, the inventors developed new vectors that permit a screening approach combining the vast diversity of phage libraries with the power of in situ screening in the context of CAR libraries. The first step is the transfer of phage scFvs (e.g., enriched) into specially designed CAR vectors allows for multiple forms of high-throughput screening using primary T cells or Jurkat reporter systems to assess binding, function, and cross-reactivity on 10s of thousands of CARs simultaneously. Top clones can rapidly identified, sequenced, and prioritized for preclinical development. This process is shown in FIG. 3.

As discussed above, the CAR vectors encode, in a 5′ to 3′ orientation, a promoter, a first restriction enzyme site cleaved by a first restriction enzyme, a transmembrane domain and a CAR endodomain, wherein no other restriction enzyme sites for said first restriction enzyme are present in said vector. Cutting with the first restriction enzyme results in a single opening such that an scFv coding region, when ligated into the opening, produces a contiguous CAR coding region.

### VI. Examples

The following examples are included to demonstrate particular embodiments. It should be appreciated by those of skill in the art that the techniques disclosed in the examples that follow represent techniques discovered by the inventor to function well in the practice of embodiments, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Results

Please refer to the attached figures and accompanying figure legends above for a discussion of the experimental details of the disclosure.

All of the compositions and methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A chimeric antigen receptor (CAR) expression vector encoding, in a 5′ to 3′ orientation, a promoter, a first restriction enzyme site cleaved by a first restriction enzyme, a single chain variable region antigen receptor, a second restriction enzyme site cleaved by said first restriction enzyme, a transmembrane domain and a CAR endodomain, wherein no other restriction enzyme sites for said first restriction enzyme are present in said vector.

2. A chimeric antigen receptor (CAR) expression vector encoding, in a 5′ to 3′ orientation, a promoter, a first restriction enzyme site cleaved by a first or second restriction enzyme, a transmembrane domain and a CAR endodomain, wherein no other restriction enzyme sites for said first restriction enzyme are present in said vector.

3. The expression vector of claim 1, further comprising a flexible linker coding region between said second restriction enzyme site and said transmembrane domain.

4. The expression vector of claim 2, further comprising a flexible linker coding region between said first restriction enzyme site and said transmembrane domain, such as CD8 hinge region.

5. The expression vector of claim 1, wherein the first restriction enzyme is Sfi1 or other unique enzyme.

6. The expression vector of claim 1, wherein said promoter is an EF1α promoter.

7. The expression vector of claim 1, wherein said hinge/transmembrane domain is derived from CD8α or CD28.

8. The expression vector of claim 1, wherein the endodomain comprises signaling domains from CD3ζ and/or 4-1BB (CD137), CD28, or any other T cell co-stimulatory domain.

9. The expression of vector of claim 1, wherein the expression vector further comprises an origin of replication.

10. The expression vector of claim 1, wherein the expression vector further comprises a CD8 leader sequence 5′ to said first restriction enzyme site and 3′ to said promoter.

11. A method of screening a immune receptor library for binding activity comprising:
(a) providing an immune receptor library;
(b) depleting said immune receptor library of non-specific pMHC binders;
(c) enriching said immune receptor library for binding to target antigen;
(d) subcloning the immune receptor regions from positive binders selected in step (c) into an expression vector according to claim 2-9 to produce a chimeric antigen receptor (CAR) library;
(e) introducing the CAR library into mammalian host cells;
(f) culturing the CAR library of step (e) under conditions supporting expression of encoded CARs;
(g) incubating the host cells of step (e) with target and off-target antigen and HLA-matched tissues;
(h) co-culturing the host cells of step (g) with on- and off-target cells (pMHC targets, HLA matched tissue, for membrane proteins, isogenic lines+/−target expression);
(i) sorting positive host cells exhibiting selective CAR binding and activation; and optionally;
(j) sequencing positive host cells.

12. The method of claim 11, wherein the target antigen is a peptide presented on MHC or membrane protein, such as one that may or may not be mutated, and that may be presented by non-classic MHC (e.g., MR1).

13. The method of claim 11, wherein the host cell is an immune effector cell.

14. The method of claim 11, wherein the host cell expresses a T cell receptor.

15. The method of claim 11, wherein the host cell is a Jurkat cell with or without NFAT or NF-kB-driven reporters, or a primary T cell.

16. The method of claim 11, wherein the host cells express a fluorescent/luminescent marker upon T cell receptor activation.

17. The method of claim 11, wherein the host cells express green fluorescent protein (GFP) or luciferase upon CAR activation.

18. The method of claim 11, wherein step (c) comprises enriching said immune receptor library for binders using matched and decoy pMHCs, optionally enriching a second, third or fourth time.

19. The method of claim 11, wherein step (c) comprises enriching said immune receptor library for binders using screening against a membrane protein target, optionally enriching a second, third or fourth time.

20. The method of claim 11, the expression vector may further comprise a flexible linker coding region between said first restriction enzyme site and said transmembrane domain, such as CD8 or CD28 hinge and transmembrane region.

21. The method of claim 11, wherein the first restriction enzyme is Sfi1 or other unique enzyme.

22. The method of claim 11, wherein said promoter is an EF1α promoter.

23. The method of claim 11, wherein said transmembrane domain is derived from CD8α.

24. The method of claim 11, wherein the endodomain comprises signaling domains from CD3ζ and 4-1BB (CD137), orCD28, ICOS, Zap70, SLP76 or other T cell signaling domains.

25. The method of claim 11, wherein the expression vector further comprises an origin of replication.

26. The method of claim 11, wherein the expression vector further comprises a CD8 leader sequence 5′ to said first restriction enzyme site and 3′ to said promoter.

27. The method of claim 11, further comprising, prior to step (a), producing said immune receptor phage library or immune receptor yeast display library.

28. The method of claim 11, further comprising performing single-cell functional assays on the sorted positive host cells of step (g).

29. The method of claim 11, wherein step (g) comprises incubating the host cells of step (d) with cells presenting the target antigen.

30. The method of claim 11, wherein step (h) comprises sorting host cells that are positive for on-target cell killing and negative for off-target cell killing.

31. The method of claim 11, further comprising sequencing the immune receptor from host cells exhibiting activated T cell receptors.

32. The method of claim 11, wherein step (c) comprises enzyme-linked immunosorbent assays.

33. The method of claim 11, wherein said immune library comprises at least 10¹⁰ unique binding sequences.

34. The method of claim 11, wherein the immune receptor library comprises scFv, Vhh, Fab, monobodies, affibodies, or nanobodies.

35. The method of claim 11, wherein the immune receptor library is synthetic or naïve.

36. The method of claim 11, wherein the method is a lossless, high-throughput screening method.

37. The method of claim 11, wherein rare immune receptors are identified.

38. The method of claim 11, comprising extension PCR with non-pComb vectors to introduce restriction sites to the immune receptor library.

39. The method of claim 11, wherein the immune receptor library is derived from a primary B cell population and the target antigen is a cancer or autoimmune target.

40. The method of claim 11, wherein the immune receptor library is generated in single-cell droplets coupling heavy and light antibody chains through overlap PCR, optionally employing primers comprising one or more restriction sites compatible with CAR library vector ligation.
